(19) United States
(12) Patent Application Publication
(10) Pub. No.: US 2008/0089947 A1
(43) Pub. Date: Apr. 17, 2008

(54) Calcium Influx Inhibitors in the Treatment of Ischemia

(76) Inventors: Clayton D. Knox, Philadelphia, PA (US); Ravi S. Chari, Nashville, TN (US); Ian B. Nicoud, Seattle, WA (US); C. Wright Pinson, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 11/840,949

(22) Filed: Aug. 18, 2007

Related U.S. Application Data
(60) Provisional application No. 60/822,836, filed on Aug. 18, 2006.

(51) Int. Cl. A61K 33/24; A01N 1/02; A61P 9/00; A61P 9/10; A61K 31/69

(52) U.S. Cl. 424/617; 435/1.1; 514/64

(57) ABSTRACT

The present invention concerns the use of calcium influx inhibitors for treatment of organs and tissues to inhibit damage caused by ischemia/reperfusion events such as transplant, other surgeries, and trauma. It includes methods and apparatuses for achieving stasis in tissue, so as to preserve and/or protect them. In specific embodiments, preservation methods and apparatuses for preserving tissue for transplantation purposes is provided.

The present application claims benefit of U.S. Provisional Application Ser. No. 60/822,836, filed Aug. 18, 2006, the entire contents of which are hereby incorporated by reference.

This invention was made with government support under grant DK-59390-1 awarded by the National Institute of Diabetes and Digestive and Kidney Diseases, and grant CA-68485-07 awarded by the National Cancer Institute. The government has certain rights in the invention.

### BACKGROUND OF THE INVENTION

1. Field of the Invention

The present invention relates generally to the fields medicine and cell biology. More particularly, it concerns methods for reducing acute ischemia and reperfusion (ischemia/reperfusion) injury in tissues and organs using a calcium influx inhibitor. In particular, the methods address ischemia/reperfusion in the settings of organ transplantation, surgery, and systemic shock, and involve the use of 2-aminoethoxydiphenyl borate (2-APB), ruthenium red, Ru360, U-73122 or analogs or derivatives of any of these parent compounds.

2. Description of Related Art

The first successful kidney transplant was performed in 1954, and the first heart was transplanted in 1967. Dr. Thomas Starzl performed the first liver transplant in 1963. Despite several decades of surgical experience and increased awareness of the need for organ donation, waiting lists remain long and one person dies every 3 hours while waiting for an organ. Due to a variety of causes, these patients may need a heart, lung, kidney, liver, or even combinations of multiple organs. Other patients are awaiting a pancreas or a cornea. While there is a constant need for organ donors, a significant hurdle in providing those in need of a transplant with a suitable organ is the limitations in current organ preservation techniques. For example, it is widely believed that a human heart must be transported within four hours for there to be any chance of the subsequent transplantation to be a success.

A key factor in the success of an organ transplant is to minimize ischemia/reperfusion damage caused by the absence of blood flow to the transplanted tissue. In order to address this problem, two methods for preserving/transporting organs for transplantation, hypothermic storage and continuous perfusion, have been developed. In the former method, the organ is removed from the donor and then rapidly cooled and transported in cold storage. In the latter method, the following steps are typically employed: 1) pulsatile flow; 2) hypothermia; 3) membrane oxygenation, and 4) a perfusate. More recently, continuous warm perfusion techniques have been explored. However, despite improvements in these methods, there remain considerable limitations in transplantation techniques and the successes flowing therefrom.

To improve the prospect of a successful transplant, techniques for better preserving an organ for transplantation have been developed. Two general areas of development have occurred, one in the area of preservation solutions and the other in the area of organ containers. No effective drugs are available to protect organs from ischemia/reperfusion during transplantation.

Ischemia/reperfusion injury is also a problem in other areas of medicine, and is not restricted to the field of transplantation. Performing surgery on organs such as the liver, heart, and brain may involve the interruption of blood flow to parts of the organ in order to minimize bleeding during the operation. When blood flow is restored, organs may be damaged by ischemia/reperfusion injury through the same biochemical mechanism responsible for this type of injury in organ transplantation. There are no effective preemptive medical treatments available to avoid such injury.

Systemic shock, a medical condition characterized by insufficient oxygen to meet the demands of the body, may also be associated with profound ischemia/reperfusion injury when the cardiopulmonary system is stabilized. There are four types of shock discussed in the medical literature: cardiogenic (associated with heart failure), hypovolemic (associated with insufficient blood volume as in, for example, traumatic injury), obstructive (associated with, for example, a pulmonary embolus), and distributive (associated with, for example, septic shock). All are associated with low blood pressure and poor blood perfusion/oxygenation of tissues. When the circulatory system is stabilized and blood pressure, blood volume, and blood oxygen carrying capacity are restored, the organs are susceptible to reperfusion injury. There is currently no pharmacologic agent available that has shown significant protection against ischemia/reperfusion injury in this setting.

### SUMMARY OF THE INVENTION

Thus, in accordance with the present invention, there is provided a method of reducing acute ischemia/reperfusion injury comprising (a) contacting a tissue or organ with a calcium influx inhibitor prior to transplantation; and (b) transplanting said tissue or organ into a recipient. The calcium influx inhibitor may be 2-APB, ruthenium red, Ru360, U-73122, or analogs or derivatives of any of these parent compounds. The calcium influx inhibitor may be any compound with the ability to inhibit mitochondrial calcium influx, either by interacting with a mitochondrial calcium influx channel directly (direct inhibition) or by interacting with another protein or molecule that is directly involved in regulating the activity of a mitochondrial calcium influx channel (indirect inhibition). The method may further comprise the step of removing said tissue or organ from a donor. The contacting may take place prior to removing, after removing or both prior to and after removing. The method may further comprise cold or warm preservation of said tissue or organ. The contacting may take place prior to cold or warm preservation, during cold or warm preservation, or both prior to and during cold or warm preservation.

The donor or recipient may be a human or a non-human mammal. The tissue may be skin, bone, bone marrow, cartilage, cornea, skeletal muscle, cardiac muscle, cardiac valve, smooth muscle, blood vessel, a limb, or a digit. The organ may be a kidney or portion thereof, a liver or portion thereof, a heart or portion thereof, a pancreas or a portion thereof, a bowel or portion thereof, or a lung or portion thereof.

The method may further comprise contacting said tissue or organ with 2-APB, ruthenium red, Ru360, U-73122, or analogs or derivatives thereof following transplantation. The method may further comprise contacting said tissue or organ with any compound with the ability to directly or indirectly inhibit mitochondrial calcium influx after transplantation. The method may further comprise administering an immunosuppressive agent to said recipient following transplantation. The calcium influx inhibitor may be administered systemically or into the vasculature of the tissue or organ. Alternatively, the tissue or organ may be immersed in a storage solution containing the calcium influx inhibitor.

In another embodiment, there is provided a method of reducing acute ischemia/reperfusion injury during interruption of circulation to a tissue or organ to facilitate a surgical procedure, comprising (a) contacting a tissue or organ with a calcium influx inhibitor; (b) interrupting the circulation to facilitate a surgical procedure; (c) performing a surgical procedure on said tissue or organ; and (d) restoring circulation to said tissue or organ.

The contacting may take place prior to the interruption of circulation, during the interruption of circulation, or after the interruption of circulation. The contacting may also take place prior to and during, during and after, prior to and after the interruption of circulation, or prior to, during and after the interruption of circulation. The calcium influx inhibitor may be 2-APB, ruthenium red, Ru360, U-73122, or analogs or derivatives thereof, or any direct or indirect inhibitor of mitochondrial calcium influx. The recipient may be a human or a non-human mammal. The calcium influx inhibitor may be administered systemically or into the vasculature of the tissue or organ.

In yet another embodiment, there is provided a method of reducing acute ischemia/reperfusion injury following sustained systemic shock to a subject comprising contacting a tissue or organ in said subject with a calcium influx inhibitor. The types of shock contemplated for use of calcium influx inhibitors include, but are not limited to cardiogenic, hypovolemic, obstructive, and distributive shock. The contacting may take place prior to systemic shock, during systemic shock, after systemic shock, prior to and during, during and after, prior to and after systemic shock, or prior to, during and after systemic shock. The calcium influx inhibitor may be 2-APB, ruthenium red, Ru360, U-73122, or analogs or derivatives thereof, or any inhibitor of mitochondrial calcium influx. The recipient may be a human or non-human mammal. The calcium influx inhibitor may be administered systemically or into the vasculature of the tissue or organ.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows mitochondrial calcium uptake after 30 minute incubation of isolated mitochondria in 37° C. incubation buffer containing increasing concentrations of 2-APB from 0-100 μM. Mitochondrial calcium uptake in the presence of incubation buffer and energy substrates pyruvate and malate (and the absence of 2-APB) was taken as baseline and considered 0% inhibition. The percentage decrease in mitochondrial calcium uptake in the presence of 2-APB is represented on the y-axis versus the log of the micromolar dose of 2-APB. The EC₅₀ is 2-3 μM.

FIG. 2 shows serum aspartate aminotransferase (AST(□)), alanine aminotransferase (ALT(▪)), and lactic acid dehydrogenase (LDH) in rats. All animals were placed under general anesthesia and underwent midline laparotomy. The animals in group 1 underwent sham operation with venous dissection and bowel manipulation, but no liver ischemia/reperfusion was induced. After one hour, animals were closed, awaken for three hours, and then serum AST, ALT, and LDH were measured. The animals in group 2 underwent the same procedure, except that a portal vein injection of DMSO (vehicle control) was given after the sham operation. The animals in group 3 underwent the same procedure, except that a portal vein injection of 2-APB (2 mg/kg in DMSO) was given after the sham operation. Group 1 establishes the normal serum marker levels for animals undergoing a sham operation, while groups 2 and 3 demonstrate that neither 2-APB nor the vehicle, DMSO, were damaging to the liver. Following the venous dissection performed as in the above groups, animals in group 4 underwent one hour of complete ischemia to the superior 70% of the liver by having an atraumatic vascular clamp placed across the branches of the portal vein and hepatic artery supplying the median and left liver lobes. After one hour of ischemia, the clamp was removed and the liver was allowed to reperfuse for three hours, during which time the animals were conscious. Animals in group 5 underwent the exact same procedure as those in group 4, except that a portal vein injection of DMSO was given immediately before the vascular clamp was placed. Animals in group 6 underwent the exact same procedure as those in group 5, except that a portal vein injection of 2-APB (2 mg/kg in DMSO) was given instead of DMSO immediately before the vascular clamp was placed. *p<0.05 vs. corresponding value in group 2; +p<0.05 vs. corresponding group 5 value and p>0.05 vs. corresponding group 3 value.

FIG. 3 shows serum transaminases (AST(□) and ALT(▪)) and lactic acid dehydrogenase (LDH) in rats. All animals were placed under general anesthesia and underwent midline laparotomy. Following dissection of the portal vein and hepatic artery, animals in group 1 underwent one hour of complete ischemia to the superior 70% of the liver by having an atraumatic vascular clamp placed across the branches of the portal vein and hepatic artery supplying the median and left lobes of the liver. After one hour of ischemia, the clamp was removed and a portal vein injection of DMSO (vehicle control) was given immediately. The liver was allowed to reperfuse for three hours, during which time the animals were conscious. Serum AST, ALT, and LDH (markers for liver damage) were then measured. Animals in group 2 underwent the exact same procedure as those in group 1, except that 2-APB (2 mg/kg in DMSO) was given instead of DMSO immediately after the clamp was removed. *p<0.05 vs. corresponding value in group 1.

FIGS. 4A-C shows black and white rendered images from rat livers magnified 400×. (FIG. 4A) Normal rat liver. Cells are healthy and intact; distinct, large, round nuclei are well-visualized. (FIG. 4B) Liver from a rat receiving DMSO injection and undergoing one hour of liver ischemia and three hours of conscious reperfusion. Greater than 90% of cells are undergoing cell death. Nuclei are shrunken, fragmented, and in some cases completely absent. A small group of surviving cells with more normal nuclei can be seen in the upper right corner. (FIG. 4C) Liver from a rat receiving portal venous injection of 2-APB (2 mg/kg in DMSO) prior to undergoing one hour of liver ischemia followed by three hours of conscious reperfusion. Cells are healthy and appear identical to the normal liver cells in FIG. 4A.

FIG. 5 shows mitochondrial calcium uptake after 30 minute incubation of isolated mitochondria in 37° C. incubation buffer containing 0.2 μM ⁴⁵Ca²⁺. Calcium uptake in the presence of incubation buffer and energy substrates pyruvate and malate was taken as baseline (100%). Upon the addition of 10 μM ruthenium red, mitochondrial calcium uptake was completely blocked (*p<0.001 vs baseline).

FIG. 6 shows serum transaminases (AST and ALT) in rats. All animals were placed under general anesthesia and underwent midline laparotomy. A portal vein injection of NaCl (vehicle, ▪) or ruthenium red (30 mg/kg in NaCl, □) was given 30 minutes prior to the induction of ischemia to the liver. Following the venous dissection performed as in the animals described in legends for FIGS. 2 and 3, animals underwent one hour of complete ischemia to the superior 70% of the liver by having an atraumatic vascular clamp placed across the branches of the portal vein and hepatic artery supplying the left and median lobes of the liver. After one hour of ischemia, the clamp was removed and the liver was allowed to reperfuse for 15 minutes, one hour, three hours, or six hours, during which time the animals were conscious. The maximal reduction of injury was observed after 6 hours of reperfusion. AST: 1556±181 vs. 597±121, p=0.005; ALT: 1118±187 vs. 294±39, p=0.005. *p<0.05, #p<0.01.

FIG. 7 shows a summary of TUNEL, and assay for apoptotic cells (programmed cell death), data for animals described in the legend for FIG. 7. Cellular apoptosis was first observed fifteen minutes following reperfusion, and the rate of apoptosis peaked after 3 hours of reperfusion. Ruthenium red (RR) significantly decreased the rate of apoptosis at all reperfusion time points measured in this study. @p=0.029, #p=0.003, *p=0.014, ‡p=0.024.

FIG. 8 shows a summary of necrosis (externally induced cell death) data for animal described in legends to FIGS. 6 and 7. Necrosis was first seen after 3 hours of reperfusion at which time the fields examined had an average of 22±4% necrosis. The percent area necrosis increased to 26%±8% by 6 hours of reperfusion (▪). As with apoptosis, ruthenium red (RR, □) pretreatment significantly decreased the observed amount of liver necrosis. *p=0.006, #p=0.026.

FIG. 9 shows western blot analyses demonstrating that isolated mitochondrial membranes (MM) were free of heavy and light plasma membrane fraction, as well as endoplasmic reticulum contamination. Prohibitin, a mitochondrial protein, was enriched in mitochondrial membranes. Positive controls (pos.) for each protein were as follows: Na/K-ATPase and alkaline phosphatase, liver plasma membranes; calreticulin, brain whole cell; prohibitin, liver whole cell.

FIG. 10 shows western blot analyses of seven PLC isoforms in rat liver whole cell (WC), mitochondrial membranes (MM), and plasma membranes (PM). PLC-δ1 was present in mitochondrial membranes, while PLC-γ1 and PLC-γ2 were found only in trace amounts that may have represented an unidentified contaminant. PLC-β1, -β2, -β3, and -ε were not found in mitochondrial membranes.

FIGS. 11A-B show mitochondrial calcium uptake after 30 minute incubation of isolated mitochondria in 37° C. incubation buffer containing pyruvate, malate, and 0.2 μM ⁴⁵Ca²⁺. Uptake in the presence of 0.14% DMSO (vehicle) was taken as baseline. (FIG. 11A) Phospholipase C inhibitor U-73122 dose-dependently inhibited mitochondrial calcium uptake when added to the incubation buffer at 0.5-7.5 μM concentrations. (FIG. 11B) U-73343, the inactive analog of U-73122, did not significantly affect mitochondrial calcium uptake when tested using the same concentration range.

FIGS. 12A-B show calcium uptake in isolated liver mitochondria with 0.2 μM extra-mitochondrial ⁴⁵Ca²⁺. (FIG. 12A) The four bars depict calcium uptake in mitochondria isolated immediately after perfusion of the liver with 4° C. UW solution. Each bar represents uptake in a separate reaction tube containing incubation buffer with the indicated compounds either present or absent. The active phospholipase C inhibitor (U-73122) significantly decreased calcium uptake (p<0.001), while the inactive form (U-73343) did not. (FIG. 12B) The same experimental conditions in (FIG. 12A) were carried out on the second half of each liver after 24 hour ischemic cold storage at 4° C. in UW solution. Calcium uptake in the presence of ATP increased significantly (p<0.001), and U-73343 again had no effect. U-73122 significantly decreased mitochondrial calcium uptake (p<0.001). Of note is that U-73122 decreased mitochondrial calcium uptake more significantly following cold ischemia than it did in mitochondria from non-ischemic livers (85±2% vs. 54±5% decrease, respectively, p<0.001), and even the raw values were lower (p<0.01). Ruthenium red also completely blocked calcium uptake in mitochondria from both ischemic and non-ischemic livers, indicating that all observed mitochondrial calcium uptake was occurring through the mitochondrial calcium uniporter. (*p<0.001 vs. ATP alone under same preparatory conditions; ♦p<0.01 vs. corresponding non-ischemic sample).

### DESCRIPTION OF ILLUSTRATIVE EMBODIMENTS

### I. Ischemia and Reperfusion Injury

As discussed above, a key limiting factor in the success of orthotopic organ transplantation and the treatment of surgical injury and systemic shock is the minimizing of ischemia/reperfusion damage caused by the absence of blood flow or oxygen to the affected tissues or organs. In fact, restarting blood flow after more than about ten minutes of ischemia is typically more damaging than the ischemia itself.

During ischemia, the lack of oxygen available to fuel cellular respiration produces a significant ATP deficit in the cell (Scott et al., 1980), resulting in bioenergetic failure and large scale membrane depolarization. These events cause a tremendous increase in cytosolic calcium to concentrations that can become toxic to the cell (both by release from intra-cellular inositol triphosphate-regulated calcium stores and by influx across the plasma membrane via store-operated calcium channels). Although the pathway through which this increase in cytosolic calcium ultimately results in cell death is not fully understood, mitochondrial calcium handling has emerged as a central process (Amberger et al., 2001).

In an attempt to rescue the cell from the ischemia/reperfusion-induced calcium spike, mitochondria sequester cytosolic calcium through the mitochondrial calcium uniporter (Amberger et al., 2001), a highly selective inner-mitochondrial membrane calcium channel that functions as the major point of entry for calcium into the mitochondria (Kirichok et al., 2004). Although this phenomenon may be of transient benefit to the cell, it decreases mitochondrial transmembrane potential, and mitochondrial calcium overload will eventually result.

Mitochondrial calcium overload represents a point of no return for the cell, after which survival following reperfusion is unlikely (Zamzami et al., 1995). At these pathophysiologic concentrations of mitochondrial calcium, a large channel known as the mitochondrial permeability transition pore is formed, and mitochondrial transmembrane potential is dissipated. This dissipation causes significant mitochondrial swelling and the release of cytochrome c and other pro-apoptotic factors from mitochondria into the cytosol (Lorenzo et al., 1999), where they initiate apoptotic pathways that lead directly to programmed cell death (apoptosis) (reviewed by Wolf and Green, 1999).

In accordance with the invention described herein, it is proposed that the use of mitochondrial and cellular calcium influx inhibitors, at times prior to, during, or following ischemic events will reduce or prevent damage due to tissue by ischemia/reperfusion. The inhibitors may be used to treat transplanted materials to improve their quality and the success of transplantation, and also to treat injured tissue to prevent additional ischemia/reperfusion injury in other settings of this type of damage, including surgery and systemic shock. The specifics of the invention are described below.

### II. Calcium Influx Inhibitors

A variety of calcium influx inhibitors are known in the art. In a particular embodiment, the present invention contemplates the use of 2-aminoethoxydiphenyl borate (2-APB). This drug is a cell-permeable modulator of store-operated calcium channels, and is now proposed as a novel inhibitor of the mitochondrial calcium uniporter. 2-APB thus has unique therapeutic potential because it blocks both store-operated calcium channels and the mitochondrial calcium uniporter, both of which may be protective during ischemia/reperfusion injury. It has been shown to have some efficacy in preventing infarct size in the heart when combined with inositol triphosphate (Gysembergh et al., 1999). The chemical formula is C₁₄H₁₆BNO, with a molecular weight of 225.1. It is found as a white crystalline solid and is soluble in ethanol at 25 mg/ml, and in DMSO at 20 mg/ml.

Another calcium influx inhibitor is ruthenium red. This drug has been shown to block TRPV1 calcium channels. It is a capsaicin and calcium antagonist that has been used as an inhibitor of the mitochondrial calcium uniporter, and the inventors demonstrate herein that ruthenium red is able to protect against ischemia/reperfusion injury. The chemical formula is H₄₂Cl₆N₁₄O₂Ru₃ with a molecular weight of 786.4. It is found as a reddish-black solid and is soluble in water.

A third calcium influx inhibitor if Ru360. This drug is closely related to ruthenium red and contains ruthenium, but is more potent. It is an established inhibitor of the mitochondrial calcium uniporter. The chemical formula is C₂H₂₆Cl₃N₈O₅Ru₂ and the molecular weight is 550.8. It exists as a reddish-brown solid and is soluble in water.

Another calcium influx inhibitor is 1-[6-((17b-3-Methoxyestra-1,3,5(10)-trien-17-yl)amino)hexyl]-1H-pyrrole-2,5-dione (U-73122). U-73122 is a selective negative antagonist of phosphatidyl inositol-specific phospholipase C. The inventors demonstrate herein the presence of phospholipase c in mitochondria (recently published, Knox et al., 2004), and show for the first time that mitochondrial phospholipase C-δ1 is upregulated and governs mitochondrial calcium uptake in the setting of ischemia/reperfusion. Thus, inhibition of mitochondrial phospholipase C with U-73122 or other inhibitors of phospholipase C or related signaling enzymes/proteins represents a novel strategy in the inhibition of mitochondrial calcium uptake. The protective effect of U-73122 was recently demonstrated through an unknown mechanism and site of action in the heart (Asemu et al., 2004), although the inventors reveal the mechanism and mitochondrial site of action herein. U-73122 exists as a white crystalline solid with the chemical formula C₂₉H₄₀N₂O₃ and a molecular weight of 464.7, and is soluble in ethanol (500 μg/ml) and DMSO (1 mg/ml).

Other calcium influx inhibitors include benzophenones such as carboxyamidotriazole, verapamil flunarizine, nifedipine, diltiazem, mibefradil, CGP-37157, and others. The following are patents and patent applications, which disclose calcium influx inhibitors: US2004/0106537; U.S. Pat. Nos. 6,699,886; 6,410,743; 5,495,005; 5,459,126; 5,051,432; 5,011,848; 4,963,571; 4,946,851; 4,935,548; 4,806,533; 4,801,599; 4,766,213; 4,504,476.

### III. Preservation Applications

The present inventors have discovered that, surprisingly, calcium influx inhibitors can be used to prevent damage to tissues and organs flowing from ischemic events and their subsequent reperfusion. The invention described herein thus comprises the use of various inhibitors of calcium influx to treat, preserve and protect tissues that will be or have been subjected to ischemia/reperfusion.

In embodiments of the invention, an organism, organ or tissue can be exposed to a calcium influx antagonist for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, or 1, 2, 3, 4, 5 or 6 weeks, and any combination or range derivable therein.

In particular embodiments, it is contemplated that an organ will be treated prior to, during, and/or after transplant. In such cases, the exposure may be conducted relative to the time of organ removal or to implantation. Again, the exposure may be initiated 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, or 1, 2, 3, 4, 5 or 6 weeks.

A. Transplanted Tissue and Organs

Various tissues and organs are contemplated as being subject to transplantation according to the present invention. In particular, the tissue may be derived from skin, bone, bone marrow, cartilage, cornea, skeletal muscle, cardiac muscle, cardiac valve, smooth muscle, blood vessels, a limb or a digit, including parts or all of the aforementioned structures. Organs include kidney, pancreas, liver, heart, lung, bowel or portions thereof. Organs for transplants may be monitored to assess their condition, particularly with respect to use as a transplant. Such methods are described in U.S. Pat. No. 5,699,793.

B. Other Preservation Agents

A variety of preservation solutions have been disclosed in which a tissue or organ is surrounded or perfused with the preservation solution for storage. One of the most commonly used solutions is ViaSpan® (Belzer UW), which employed with cold storage. Other examples of such solutions or components of such solutions include the St. Thomas solution (Ledingham et al., 1987), Broussais solution, UW solution (Ledingham et al., 1990), Celsior solution (Menasche et al., 1994), Stanford University solution, and solution B20 (Bernard et al., 1985), as well as those described and/or claimed in U.S. Pat. Nos. 6,524,785; 6,492,103; 6,365,338; 6,054,261; 5,719,174; 5,693,462; 5,599,659; 5,552,267; 5,405,742; 5,370,989; 5,066,578; 4,938,961; and, 4,798,824.

Allopurinol inhibits xanthine oxidase, blocking the conversion of xanthine and oxygen to superoxide and uric acid. Glutathione is used as an antioxidant with membrane-stabilizing properties. Dexamethasone also stabilizes membranes. Magnesium seems to counteract some of the effects of intracellular calcium and the sulfate ion resists cell swelling because it is relatively impermeable to cell membranes.

In addition to solutions, other types of materials are also known for use in transporting organs and tissue. These include gelatinous or other semi-solid material, such as those described, for example, in U.S. Pat. No. 5,736,397.

Some of the systems and solutions for organ preservation specifically involve oxygen perfusion in the solution or system to expose the organ to oxygen because it is believed that maintaining the organ or tissue in an oxygenated environment improves viability. Kuroda et al., 1988 and U.S. Pat. Nos. 6,490,880; 6,046,046; 5,476,763; 5,285,657; 3,995,444; 3,881,990; and, 3,777,507. Isolated hearts that are deprived of oxygen for more than four hours are believed to lose vigor and not be useful in the recipient because of ischemic/reperfusion injury. U.S. Pat. No. 6,054,261.

Moreover, many, if not all, of the solutions and containers for organ preservation and transplantation involve hypothermia (temperature below room temperature, often near but not below 0° C.), which has been called the “bed rock of all useful methods of organ and tissue preservation.” U.S. Pat. No. 6,492,103.

As discussed in U.S. Pat. Nos. 5,952,168, 5,217,860, 4,559,258 and 6,187,529 (incorporated specifically by reference), biological materials can be preserved, for example, for keeping transplantable or replaceable organs long-term. Cells, tissue/organs, or cadavers can be provided with compounds that enhance or maintain the condition of organs for transplantation. Such methods and compositions include those described in U.S. Pat. Nos. 5,752,929 and 5,395,314.

It is contemplated that any agent or solution used with a biological sample that is living and that will be used as a living material will be pharmaceutically acceptable or pharmacologically acceptable. The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared either as liquid solutions or suspensions; solid forms suitable for solution in, or suspension in, liquid prior to use can also be prepared. Any of the aforementioned solutions and transport strategies are considered suitable vehicles to carry a calcium influx inhibitor to the tissue or organ in question

A number of drugs can be administered to a patient after receiving an organ transplant to assist in the recovery process. Such drugs include compounds and agents that reduce or inhibit an immune response against the donated organ.

C. Preservation Apparati

Systems or containers for transporting organs and tissues have also been developed through the years. Any of these embodiments may be combined with apparati of the invention, which allow for use with oxygen antagonists.

Most involve cooling systems for implementation, for example, those described in U.S. Pat. Nos. 4,292,817, 4,473,637, and 4,745,759, which employ active refrigeration with a cooling liquid that is pumped through the system. Several sophisticated devices have been designed involving multiple chambers or dual containers, such as is U.S. Pat. Nos. 5,434,045 and 4,723,974.

Some constitute a system in which an apparatus is devised for perfusion of the organ or tissue in a preservation solution, as is described in U.S. Pat. Nos. 6,490,880; 6,100,082; 6,046,046; 5,326,706; 5,285,657; 5,157,930; 4,951,482; 4,502,295; and, 4,186,565.

Some of the systems and solutions for organ preservation specifically involve oxygen perfusion in the solution or system to expose the organ to oxygen because it is believed that maintaining the organ or tissue in an oxygenated environment improves viability. Kuroda et al., 1988; U.S. Pat. Nos. 6,490,880; 6,046,046; 5,476,763; 5,285,657; 3,995,444; 3,881,990; and, 3,777,507. Isolated hearts that are deprived of oxygen for more than four hours are believed to lose vigor and not be useful in the recipient because of ischemic/reperfusion injury. U.S. Pat. No. 6,054,261.

### IV. Surgery and Systemic Shock

Recent advances in the field of surgery have allowed surgeons to perform procedures that were considered impossible 30 years ago. Advances in operative management and surgical and imaging modalities, among many other factors, have paved the way for more aggressive approaches to surgical care. However, many of the operations performed every day carry significant risk, especially when blood flow must be altered to accommodate a surgical procedure.

Performing surgery on the liver, kidney, heart, brain, and other organs often requires that the surgical team stop blood flow to an organ or tissue to prevent massive bleeding and the possibility of terminal organ damage or exsanguination. Although the lack of blood and thus lack or oxygen may be tolerated by bodily tissue for variable durations, organs may suffer significant injury during the ischemic period, and especially during reperfusion following the surgical procedure. In light of the findings disclosed by the current inventors herein, it is proposed that cellular and mitochondrial calcium influx inhibitors be used in the treatment of subjects undergoing a surgical procedure in order to protect against ischemia/reperfusion injury during any surgical procedure that involves alteration of blood flow to an organ or tissue in order to facilitate said surgical procedure.

Systemic shock is another situation encountered frequently by health care providers. Shock is defined as a physiologic state in which the body is unable to deliver sufficient oxygen to meet the demands of the body. Under normal conditions, oxygen delivery is five times higher than oxygen demand, creating a significant excess of oxygen. During shock, demand increases and supply decreases to the point that there is more oxygen consumption than delivery, resulting in hypoxia. Shock may be caused by cardiac insufficiency (cardiogenic shock, as in chronic heart failure, acute myocardial infarction, or following cardiac surgery), hypovolemia (as in hemorrhage, trauma, or dehydration), obstruction (as in pulmonary embolism or pericardial tamponade), sepsis, anaphylaxis, or spinal shock. Unintentional traumatic injury is an especially important cause of systemic shock, and is the number one killer of children and adolescents across the globe. Regardless of the etiology, systemic shock is characterized by low blood pressure and an inability of the circulatory system to deliver sufficient oxygen to meet the demands of the body. Unfortunately, by the time the circulatory system is stabilized, many patients have already sustained significant ischemic injury to their organs. The brain, kidneys, liver, bowel, and almost all other organs and tissues may be affected to some degree by such injury. The subsequent restoration of normal blood flow further subjects these organs and tissues to reperfusion injury, which can cause organ failure due to massive cell death.

The only way to prevent such injury is to not reperfuse, which is not compatible with life, and there is thus a need for efficacious drugs capable of ameliorating the ischemia/reperfusion injury in subjects experiencing systemic shock. It is proposed that a calcium influx inhibitor could be administered to a subject experiencing systemic shock in order to protect the subject's organs and tissues from ischemia/reperfusion injury.

### V. Modes of Administration and Pharmaceutical Compositions

An effective amount of a calcium influx inhibitor pharmaceutical composition, in the context of the present invention, is defined as that amount sufficient to improve the quality of transplanted material, or to reduce ischemia/reperfusion injury in tissues or organs with insufficient blood or oxygen delivery. More rigorous definitions may apply, including successful transplantation or protection of injured/damaged tissue from ischemic injury.

A. Exposure

The routes of administration of a calcium influx inhibitor will vary, naturally, with the cell type and tissue/organ location; however, generally cells/tissues/organs will be exposed to a calcium entry antagonist by incubating them with a calcium influx inhibitor (which may be a gas, liquid, or semi-solid liquid), immersing them in the calcium influx inhibitor (which may be a liquid or semi-solid liquid), injecting them with the calcium influx inhibitor (which may be a gas, liquid or semi-solid liquid), or perfusing them with the calcium influx inhibitor (which may be a liquid or semi-solid liquid). When the calcium influx inhibitor is a gas, it is contemplated that the gas may be blown onto the cells, or the cells may be exposed to the gas in a closed or significantly closed container or chamber.

Apparati discussed herein can be used to expose cells/tissues/organs to a calcium influx inhibitor. It is contemplated that the calcium influx inhibitor can be cycled in and out of a chamber or container in which the cells/tissues/organs are housed, or that the amount of the calcium influx inhibitor to which the cells are exposed can vary periodically or intermittently.

B. Formulations

Solutions of the active compounds may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, DMSO, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15^{th} Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions are prepared by incorporating the active compounds in the required amount in the appropriate solvent with various combinations of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

C. Perfusion Systems

A perfusion system for cells/tissues/organs may be used to expose materials to a calcium influx inhibitor in the form of a liquid or a semi-solid. Perfusion refers to continuous flow of a solution through or over a population of cells. It implies the retention of the cells within the culture unit as opposed to continuous-flow culture, which washes the cells out with the withdrawn media (e.g., chemostat). Perfusion allows for better control of the culture environment (pH, pO₂, nutrient levels, calcium influx inhibitor levels, etc.) and is a means of significantly increasing the utilization of the surface area within a culture for cell attachment.

The technique of perfusion was developed to mimic the cells milieu in vivo where cells are continuously supplied with blood, lymph, or other body fluids. Without perfusion of a physiological nutrient solution, cells in culture go through alternating phases of being fed and starved, thus limiting full expression of their growth and metabolic potential. In the context of the present invention, a perfusion system may also be used to perfuse cells/tissues/organs with a calcium influx inhibitor.

Those of skill in the art are familiar with perfusion systems, and there are a number of perfusion systems available commercially. Any of these perfusion systems may be employed in the present invention. One example of a perfusion system is a perfused packed-bed reactor using a bed matrix of a non-woven fabric (CelliGen™, New Brunswick Scientific, Edison, N.J.; Wang et al., 1992; 1993; 1994). Briefly described, this reactor comprises an improved reactor for culturing of both anchorage- and non-anchorage-dependent cells. The reactor is designed as a packed bed with a means to provide internal recirculation. Preferably, a fiber matrix carrier is placed in a basket within the reactor vessel. A top and bottom portion of the basket has holes, allowing the medium to flow through the basket. A specially designed impeller provides recirculation of the medium through the space occupied by the fiber matrix for assuring a uniform supply of nutrient and the removal of wastes. This simultaneously assures that a negligible amount of the total cell mass is suspended in the medium. The combination of the basket and the recirculation also provides a bubble-free flow of oxygenated medium through the fiber matrix. The fiber matrix is a non-woven fabric having a “pore” diameter of from 10 μm to 100 μm, providing for a high internal volume with pore volumes corresponding to 1 to 20 times the volumes of individual cells.

The perfused packed-bed reactor offers several advantages. With a fiber matrix carrier, the cells are protected against mechanical stress from agitation and foaming. The free medium flow through the basket provides the cells with optimum regulated levels of oxygen, pH, and nutrients. Products can be continuously removed from the culture and the harvested products are free of cells and can be produced in low-protein medium, which facilitates subsequent purification steps. This technology is explained in detail in WO 94/17178 (Freedman et al.), which is hereby incorporated by reference in its entirety.

The Cellcube™ (Corning-Costar) module provides a large styrenic surface area for the immobilization and growth of substrate attached cells. It is an integrally encapsulated sterile single-use device that has a series of parallel culture plates joined to create thin sealed laminar flow spaces between adjacent plates.

The Cellcube™ module has inlet and outlet ports that are diagonally opposite each other and help regulate the flow of media. During the first few days of growth the culture is generally satisfied by the media contained within the system after initial seeding. The amount of time between the initial seeding and the start of the media perfusion is dependent on the density of cells in the seeding inoculum and the cell growth rate. The measurement of nutrient concentration in the circulating media is a good indicator of the status of the culture. When establishing a procedure it may be necessary to monitor the nutrient composition at a variety of different perfusion rates to determine the most economical and productive operating parameters.

Other commercially available perfusion systems include, for example, CellPerf® (Laboratories MABIO International, Tourcoing, France) and the Stovall Flow Cell (Stovall Life Science, Inc., Greensboro, N.C.).

The timing and parameters of the production phase of cultures depends on the type and use of a particular cell or tissue. Many cultures require a different media for production than is required for the growth phase of the culture. The transition from one phase to the other will likely require multiple washing steps in traditional cultures. However, one of the benefits of a perfusion system is the ability to provide a gentle transition between various operating phases. The perfusion system can also facilitate the transition from a growth phase to a static phase induced by a calcium influx inhibitor. Likewise, the perfusion system can facilitate the transition from a static phase to a growth phase by replacing the solution comprising a calcium influx inhibitor with, for example, a physiological nutrient media. In the field or organ transplantation, cold storage and perfusion has traditionally been the accepted approach, but warm perfusion and storage techniques are the topic of current research, and their use in combination with a calcium influx inhibitor is contemplated.

### VI. Combination Treatments

The compounds and methods of the present invention may be used in the context of a number of therapeutic applications, particularly organ transplants or traumas. In order to increase the effectiveness of a treatment with the compositions of the present invention, such as calcium influx inhibitors, it may be desirable to combine these compositions with other agents effective in the treatment of ischemia and/or other aspects of transplantation or trauma (secondary agent). For example, chemo-immunosuppressants may be administered in conjunction with transplantation. Moreover, additional drugs, such as those described in U.S. Pat. No. 6,552,083 (inhibitory agent comprising N-(3,4-dimethoxycinnamoyl)anthranililc acid) and U.S. Pat. No. 6,013,256 (antibodies that bind the IL-2 receptor, such as a humanized anti-Tax antibody) may be employed.

Various combinations may be employed; for example, a calcium influx inhibitor, such as 2-APB, ruthenium red, Ru360, or U-73122 is “A” and the secondary therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of calcium influx inhibitors of the present invention will follow general protocols for the administration of drugs and/or biologicals, taking into account the toxicity, if any, of the treatment. It is expected that the treatment cycles would be repeated as necessary.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Rationale: The experiments in Example 1 were designed to test the effect of 2-APB on mitochondrial calcium influx in isolated liver mitochondria, and to confirm previous reports that ruthenium red is a reliable inhibitor or mitochondrial calcium influx.

Methods: The methods described herein are relevant to Examples 1 and 4. Livers were isolated from male Sprague-Dawley rats (300 g). Rats were anesthetized with isoflurane and sacrificed by cervical dislocation. Whole mitochondria were separated from rat liver as previously described by Knox et al. (2004). Briefly, the liver was minced and homogenized in liver homogenization buffer (LHB)(0.2 M mannitol, 50 mM sucrose, 10 mM KCl, and 1 mM Na₂EDTA, pH=7.4, KOH). The homogenate was filtered through gauze and centrifuged 2 times for 10 min at 1,000× g. The supernatant was collected and centrifuged for 10 min at 3,000× g. The pellet was washed twice and resuspended in calcium-free LHB without EDTA to a concentration of 2 mg/mL. All steps were performed at 4° C.

All mitochondrial calcium uptake studies were performed in incubation buffer (100 mM KCl, 20 mM HEPES, 5 mM MgCl₂, pH=7.4, KOH) as previously described by Knox et al. (2004). A combination of metabolic substrates pyruvic acid and malic acid (final concentration 1 mM each) was used as a source of mitochondrial energy. Radioactive ⁴⁵Ca²⁺ (NEN Life Sciences Products, Inc., Boston, Mass.) was added to the incubation buffer (final concentration range: 0.1-2.0 μM). The experiments were initiated by addition of mitochondria to the incubation buffer (final concentration 0.2 mg/ml). 2-APB (in DMSO) was added at concentrations from 0-100 μM to determine its effects on mitochondrial calcium uptake. Mitochondria were incubated in a 37° C. shaking water bath for 30 minutes, filtered, and washed. Accumulated mitochondrial ⁴⁵Ca²⁺ was then measured using liquid scintillation counting in a Beckman LS6000IC beta counter. Mitochondria in incubation buffer without pyruvic acid and malic acid served as background controls. Ruthenium red (10 μM), a uniporter antagonist, was tested in all conditions to determine inhibitory capacity on observed mitochondrial calcium uptake, and to ensure that all observed calcium uptake was through the uniporter.

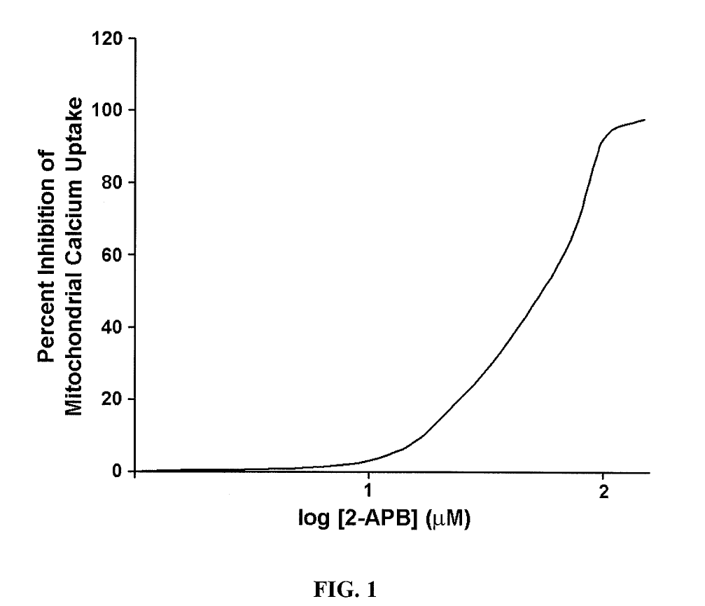

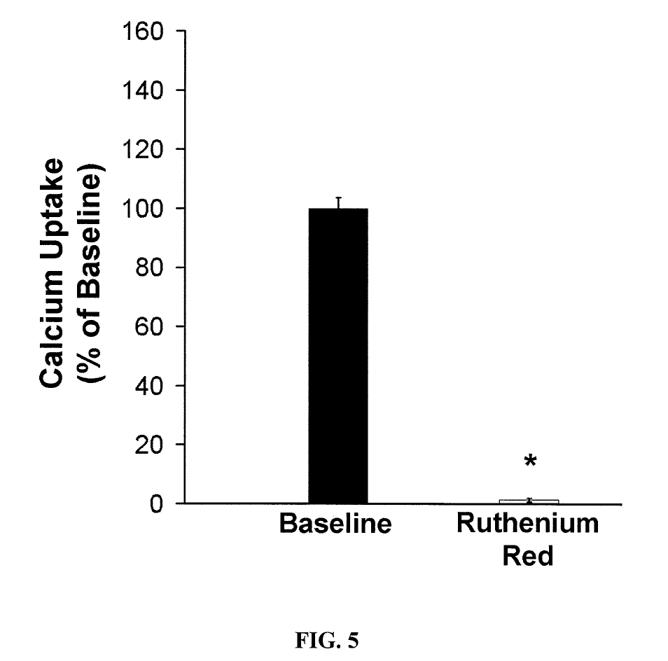

Results: Maximal ⁴⁵Ca²⁺ uptake was found to occur after 30 minutes, which was calculated to be 1-2 pm/mg mitochondrial protein/minute. This value was taken as baseline. In background controls, which contained no pyruvate or malate, uptake was less than 10% of baseline. Ruthenium red completely blocked mitochondrial calcium uptake at a concentration of 10 μM, confirming that the ⁴⁵Ca²⁺ was entering the mitochondria exclusively through the mitochondrial calcium uniporter, and that ruthenium red is a reliable inhibitor of mitochondrial calcium influx (FIG. 5). 2-APB consistently and dose-dependently inhibited mitochondrial calcium influx in the range of 1-100 μM 2-APB (FIG. 1).

Conclusions: 2-APB, a borane known to antagonize store-operated calcium channels, is a novel inhibitor of the mitochondrial calcium uniporter and mitochondrial calcium influx. Additionally, ruthenium red, as previously indicated by other authors, is also a potent inhibitor of mitochondrial calcium influx.

### Example 2

Rationale: The experiments in Example 2 were designed to test the in vivo ability of 2-APB to protect against liver ischemia/reperfusion injury, based on the data in Experiment 1 identifying 2-APB as a novel inhibitor of mitochondrial calcium influx and the hypothesis that inhibition of mitochondrial calcium influx is protective against ischemia/reperfusion injury.

Methods: Following overnight fast, male Sprague-Dawley rats were placed under general anesthesia with isoflurane gas (3%, 2 L O₂). Via a midline incision, a laparotomy was performed and the portal vein, hepatic artery, and bile duct were identified and dissected free from surrounding structures. 2-APB (2 mg/kg in DMSO) or vehicle (DMSO) was injected into the portal vein, followed immediately by 1 hour of inflow occlusion to 70% of the liver, or by sham operation (n>3/group). Approximately 70% liver ischemia was created by using an atraumatic vessel clamp (S&T Microclamp B-3, Fine Science Tools, Foster City, Calif.) to occlude the portal vein and hepatic artery branches to the median and left liver lobes. This method is widely reported and avoids mesenteric congestion by allowing portal venous drainage via the right lobe and caudate lobe. The clamp was removed after 1 hour and the livers were given three hours to reperfuse with blood while the animals were conscious. Liver injury was assessed by serum levels of AST, ALT, LDH, which are three standard serum markers of acute ischemia/reperfusion injury to the liver. Histologic liver analysis using H&E staining and TUNEL assay, which identifies terminally damaged (apoptotic) cells, were used to look directly at cell damage in the collected livers.

To investigate the cellular mechanisms of 2-APB, an in vitro model using hepG2 cells transfected with GFP-tagged cytochrome c was used. Hep G2 cells have been previously used in the study of liver regeneration and oncogenesis and ischemic injury. Cells were grown at 37° C., 5% CO₂ in MEM (ATCC, Manassas, Va.), with 10% fetal bovine serum, and 1% penicillin/streptomycin to 80% confluence. The media was replaced with UW solution and cells were incubated at 4° C. in hypoxic conditions for 6 hours. Hypoxia was achieved by placing the cells in an airtight incubator (Form a Scientifica, Marietta, Ohio), which was flushed with 5% CO₂ and 95% N₂ until the oxygen content in the container reached <0.1%, as verified using a dissolved O₂ meter (Model 4000, VWR Scientific Products, Suwannee, Ga.). To render the storage solution hypoxic before experiments were carried out, UW was preincubated in the hypoxic chamber in an open sterile container for 8 h before experiments were carried out. This resulted in a final O₂ concentration of <0.1% as measured with the dissolved O₂ meter. Following 6 hours of hypothermic storage, the UW was replaced with warm, oxygenated media, and the cells were incubated at 37° C. Cytoplasmic extracts were harvested in lysis buffer (10 mM of KCl, 10 mM of HEPES, 1 mM of NaEDTA, 200 mM of Mannitol, 50 mM of sucrose) immediately following storage and after 60, 120, and 180 min of rewarming. HepG2 cytoplasmic extracts were harvested before exposure to storage conditions to serve as a control (untreated control). In addition HepG2 cells were stored in UW under normoxic conditions at 37° C. in order to control for any effect from UW (negative control). Ruthenium red, a mitochondrial uniporter inhibitor (Sigma, St Louis, Mo.; 10 μM), was added to the UW solution of selected samples before storage of cells.

Details of Specific Methodologies: The following descriptions of specific experimental techniques are applicable to Examples 2, 3 and 4.

Serum Transaminase Determinations: Serum collected from each animal at the time of euthanasia was analyzed for aspartate aminotransferase (AST), alanine aminotransferase (ALT), and lactic acid dehydrogenase (LDH) concentrations. These measurements were carried out by Mid South Vet Lab.

Assessment of apoptosis: In order to quantitate the rate of apoptosis in a sensitive manner, the inventors performed terminal deoxynucleotidyl transferase-mediated deoxyuridine triphosphate nick-end labeling (TUNEL) on paraffin embedded tissue sections from each animal. This assay was performed following the manufacturer's recommendations for a commercially available kit (ApopTag Plus Fluorescein In Situ Apoptosis Detection Kit, Chemicon International, Inc., Temecula, Calif.). Five randomly selected fields from each tissue section were examined at an original magnification of 200×. The number of positively staining cells per field was recorded. The average number of positively staining nuclei per field for each animal was used for analysis.

Quantitation of liver necrosis: Paraffin embedded tissue sections were stained with hematoxylin and eosin (H&E) and examined at an original magnification of 200×. Digital photomicrographs were made from five randomly selected fields of each tissue section. Necrosis was defined as areas demonstrating one or more of the following characteristics: nuclear pyknosis, cytoplasmic hypereosinophilia, loss of distinct cellular borders, hemorrhage, and neutrophil infiltration. These traits correspond to grade 2 and 3 liver injury. ImagePro software was used to create specific overlays of any region of necrosis in each of the photomicrographs. Using these overlays, percent area necrosis was calculated for each photomicrograph. The average percent area necrosis for each animal was then used for analysis.

Statistical Analysis: Statistical significance between study groups was determined using a two-tailed homoscedastic student t-test. A p value of ≦0.05 was considered significant.

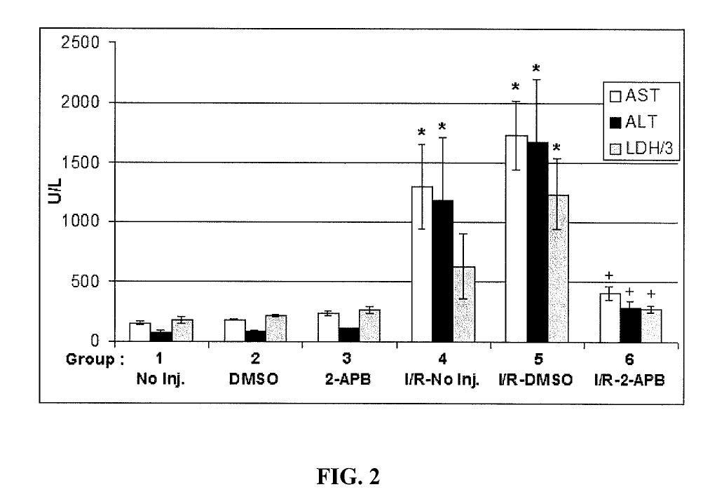

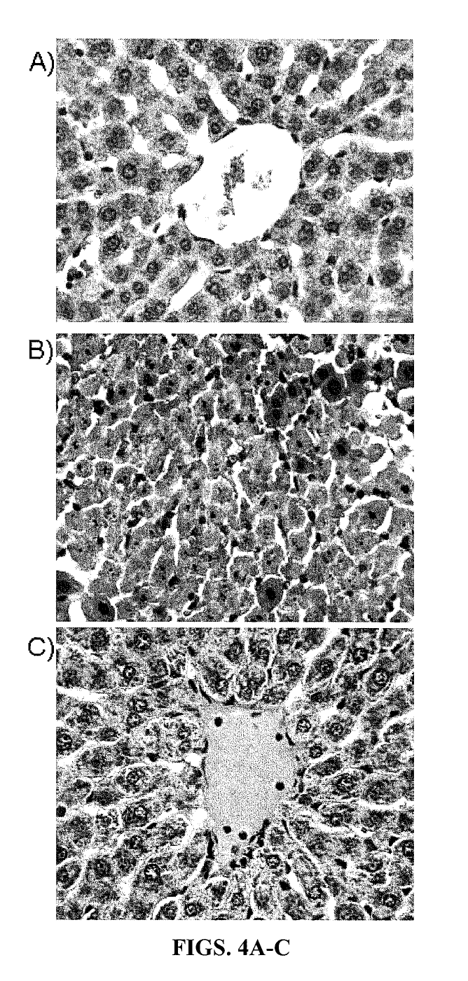

Results: 1) In ischemia/reperfusion rats receiving vehicle (DMSO) (FIG. 2, group 5), AST, ALT and LDH levels rose by 1012±163%, 2181±721%, and 644±133%, respectively, following ischemia/reperfusion (p<0.05 vs. DMSO shams (FIG. 2, group 2)). 2) Injection with 2-APB prior to ischemia/reperfusion (FIG. 2, group 6) eliminated the rise in AST, ALT and LDH (p<0.05 vs. DMSO shams (FIG. 2, group 2)). 3) Rats receiving DMSO and ischemia/reperfusion demonstrated significant liver damage by H&E and TUNEL assay, with up to 75% of liver cells per random field undergoing cell death (FIG. 4B) compared to normal rat liver (FIG. 4A). 4) Livers from ischemia/reperfusion rats receiving 2-APB had ≦2% cells undergoing cell death (FIG. 4C). 5) 6 hour ischemic hepG2 cells in University of Wisconsin solution demonstrated near complete release of GFP-tagged cytochrome c into the cytoplasm. 6) Addition of 100 μM 2-APB to UW prior to cold storage prevented this release.

Conclusion: 2-APB, when administered prior to an ischemic insult, prevents in vivo ischemia/reperfusion damage to the liver, keeping the majority of liver cells alive and functional after such an insult. The mechanism of this protection involves the prevention of cytochrome c release, as evidenced by hepG2 cells subjected to in vitro ischemia/reperfusion. These data identify 2-APB as a novel agent to minimize ischemia/reperfusion damage associated with trauma, surgery, and transplantation.

### Example 3

Rationale: Example 2 shows that 2-APB protects against liver ischemia/reperfusion injury when administered prior to ischemia, and the experiments in Example 3 were designed to determine whether or not 2-APB is also protective when administered after the ischemic event, as would be the case in situations such as shock, trauma, and other clinical scenarios involving unexpected hypoxia.

Methods: Under isoflurane anesthesia, 18 rats underwent sham operation or 1 hour of warm ischemia to the superior 70% of the liver via atraumatic portal venous and hepatic arterial clamping as described in Example 2. 2-APB (2 mg/kg in DMSO), or vehicle (DMSO) was injected into the portal vein following 1 hour of ischemia, concurrent with removal of the vascular clamp. Animals were sacrificed after 3 hours of reperfusion (n=6 per group). Serum AST, ALT, and LDH levels were measured, and liver samples were evaluated histologically using H&E and TUNEL staining. To investigate the effect of 2-APB at the cellular level, an I/R model in HepG2 cells transfected with GFP-tagged cytochrome c was used. Statistical significance was assessed using Student t-test.

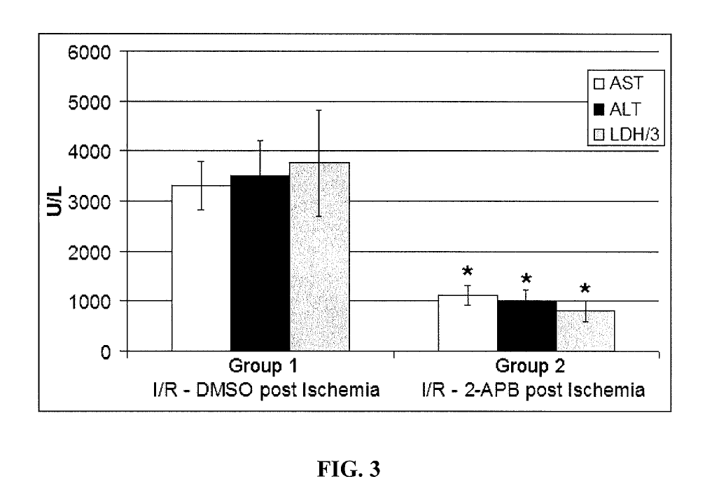

Results: 1) 1 hour of ischemia followed by 3 hours of reperfusion with DMSO significantly increased serum AST, ALT, and LDH levels by 1682+/−293%, 4030+/−603%, and 1640+/−459%, respectively, compared to DMSO shams (FIG. 3, group 1) (p<0.05 for all). 2) Administration of 2-APB at the onset of reperfusion significantly inhibited this elevation of AST, ALT, and LDH (FIG. 3, group 2) (p<0.05 for all). 3) H&E histology demonstrated little evidence of liver necrosis when 2-APB was administered during reperfusion.

Conclusions: 2-APB administration after ischemia and at the onset of reperfusion protects the liver against reperfusion injury. 2-APB is a novel agent for administration following an ischemic insult to prevent reperfusion injury to the liver.

### Example 4

Rationale: The experiments in Example 4 were designed to verify that the protective effect of 2-APB against ischemia/reperfusion injury (as demonstrated in Examples 2 and 3) involves inhibition of calcium influx. Thus, ruthenium red, another inhibitor of mitochondrial calcium influx with a completely unrelated chemical structure was tested in a similar fashion. Ruthenium red, like 2-APB, also inhibits cellular calcium influx at certain plasma membrane calcium channels.

Methods: Under isoflurane anesthesia, rats underwent sham operation or 1 hour of warm ischemia to the superior 70% of the liver via atraumatic portal venous and hepatic arterial clamping as described in Example 2. Thirty minutes prior to the initiation of liver ischemia, animals received ruthenium red (30 mg/kg in NaCl) or vehicle (0.9% NaCl) IV. Ischemia was carried out for one hour. The total anesthesia time for the animals was 90 minutes. Sham operated animals received laparotomy, mobilization of the liver, and underwent equivalent anesthesia. Following vascular clamp removal, animals were allowed to reperfuse for periods of 15 minutes, 1 hr, 3 hr, or 6 hr. At the time of animal sacrifice, serum was collected, and liver tissue was preserved for histological analysis. A total of 40 animals were used for this experiment. Ten animals were used for each time point: 4 ischemia/reperfusion, 4r ischemia/reperfusion with ruthenium red pretreatment, and 2 sham operated animals. Liver injury was assessed by serum levels of AST, ALT, LDH, which are three standard serum markers of acute ischemia/reperfusion injury to the liver. Histologic liver analysis using H&E staining and TUNEL assay, which identifies terminally damaged (apoptotic) cells, were used to look directly at cell damage in the collected livers. All experimental measurements and data were obtained as detailed in Example 2.

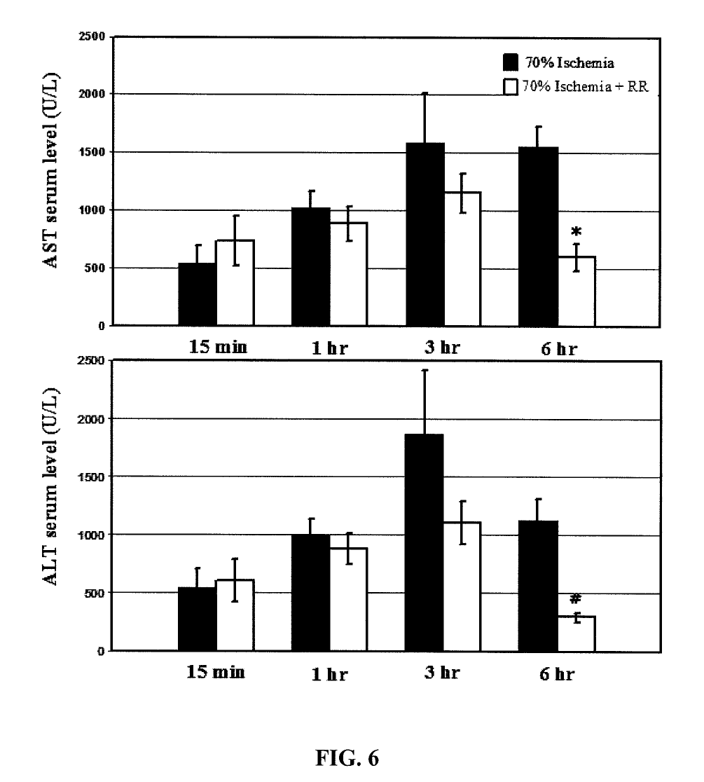

Results: Peak AST and ALT levels following 1 hour of 70% liver ischemia occurred after 3 hours of reperfusion (FIG. 6). There was a trend for the animals pretreated with ruthenium red to have lower AST and ALT levels after 3 hours of reperfusion, and the maximal effect of ruthenium red pretreatment occurred following 6 hours of reperfusion. Inhibition of mitochondrial calcium uptake with ruthenium red resulted in a 2.5-fold reduction in AST levels and a 4-fold reduction in ALT levels at the 6 hour reperfusion time point (FIG. 6). Pretreatment of sham operated animals with ruthenium red did not alter AST or ALT levels when compared to sham operated animals receiving vehicle alone (data not shown).

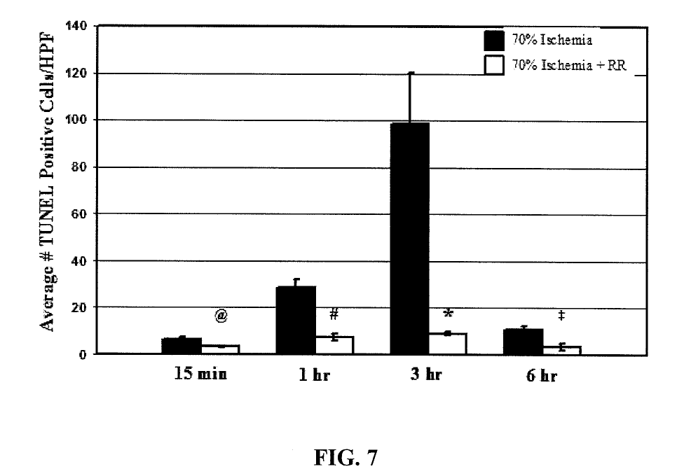

Ruthenium red significantly decreased the rate of cell death by apoptosis at all reperfusion time points measured in this study. The majority of apoptosis was noted in hepatocytes located in zone 3. The rate of apoptosis was quantified by counting the number of positively staining cells per high power field, and 5 fields were averaged for each animal. Cellular apoptosis was observed as early as fifteen minutes following reperfusion when compared to the apoptotic rates observed in the sham operated animals (6.3±1.2 cells/hpf vs. 0.8±0.2 cells/hpf, p=0.029). The rate of TUNEL positively increased after 1 hour and subsequently peaked at the 3 hour time point with 98±21 cells/hpf. The rate of apoptosis decreased by 6 hours of reperfusion; however, the levels remain significantly higher than sham (FIG. 7) (10.6±1.8 cells/hpf vs. 0.8±0.2 cells/hpf, p=0.024).

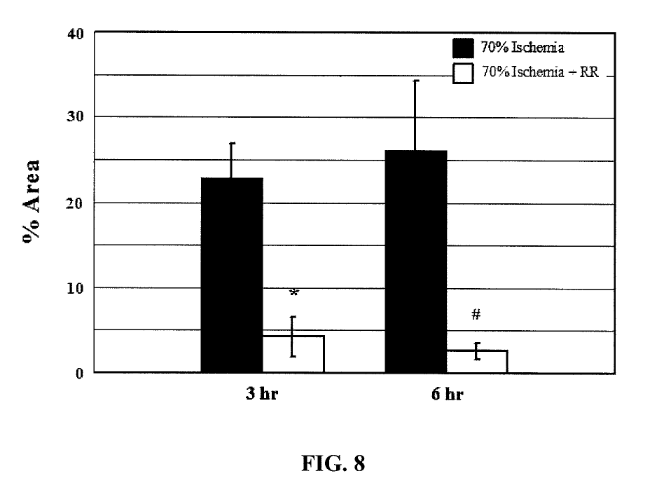

Cell death by necrosis was semiquantitated as described in the methods section and expressed as a percentage of the total tissue section. Histologic criteria for grade 2 or grade 3 injury was not seen in tissue sections taken at 15 minutes or 1 hour following reperfusion. After 3 hours of reperfusion, the fields examined had an average of 22%±4% necrosis. This increased to 26%±8% by 6 hours of reperfusion. As summarized in FIG. 8, ruthenium red pretreatment decreased the observed amount of liver necrosis to 5%±2% and 3%±1% after 3 hours and 6 hours of reperfusion respectively (p=0.006 and 0.026, respectively).

Conclusions: Ruthenium red, a selective inhibitor of the mitochondrial calcium uniporter, is protective during warm ischemia/reperfusion injury in the liver. When administered prior to the ischemic insult, ruthenium red significantly reduces the amount of ischemia/reperfusion injury as evidenced by decreased liver enzymes, decreased cellular apoptosis, and decreased tissue necrosis. Since the biochemical mechanism of injury is the same throughout the different tissues in the body, ruthenium red may thus have significant clinical utility for the treatment of ischemia/reperfusion damage in human organs and tissues.

### Example 5

Rationale: Although a calcium channel known as the mitochondrial calcium uniporter has long been known to exist as the major point of entry for calcium into the mitochondria, the biochemical machinery and mechanisms controlling mitochondrial calcium influx are unknown. Many cellular calcium channels are controlled by the signaling enzyme phospholipase C (PLC), and Experiment 4 was designed to determine whether or not PLC exists in isolated mitochondria and, if so, whether it plays a role in mitochondrial calcium influx.

Methods: Whole mitochondria were separated from rat liver as previously described in Example 1. Ultra-pure mitochondrial membranes were isolated from these mitochondria as previously described by Hagopian (Hagopian, 1999) in order to test for the presence of membrane-bound PLC in mitochondria. Plasma membranes were also isolated to serve as controls for the PLC proteins.

Western blots were performed on isolated mitochondrial membranes to determine the presence of different isoforms of PLC as described by Knox et al. (2004). Mitochondrial membrane purity was analyzed using additional western blots probed for alkaline phosphatase, Na/K ATPase, calreticulin, and prohibitin. Anti-alkaline phosphatase and anti-Na/K ATPase antibodies were used to detect heavy and light plasma membrane fraction contamination, respectively. An anti-calreticulin antibody was used to determine contamination from the endoplasmic reticulum, while anti-prohibitin antibodies were used to confirm the presence of mitochondria. Blots were probed using antibodies directed against different isoforms of PLC (PLC-γ1, -γ2, -β1, -β2, -β3, -δ1, and -ε)

Mitochondrial calcium uptake studies were performed in an incubation buffer as described in Example 1. A combination of metabolic substrates pyruvic acid and malic acid was used as a source of mitochondrial energy. Radioactive ⁴⁵Ca²⁺ was added to the incubation buffer (final concentration range: 0.1-2.0 μM). A selective PLC inhibitor, 1-[6-((17β-3-Methoxyestra-1,3,5(10)-trien-17-yl)amino)hexyl]-1H-pyrrole-2,5-dione (U-73122), was added to some samples to determine the effect of PLC inhibition on mitochondrial calcium uptake. The inactive analog of U-73122, 1-[6-((17β-3-Methoxyestra-1,3,5(10)-trien-17-yl)amino)hexyl]-2,5-pyrrolidinedione (U-73343), was used as a control since it differs from U-73122 by only a sole double bond but has no effect on PLC or other molecules involved in calcium signaling. The calcium uptake experiments were initiated by addition of mitochondria to the incubation buffer (final concentration 0.2 mg/ml). Mitochondria were incubated in a 37° C. shaking water bath for 30 minutes, filtered, and washed, and calcium uptake quantified. Mitochondria in incubation buffer without pyruvic acid and malic acid served as background controls.

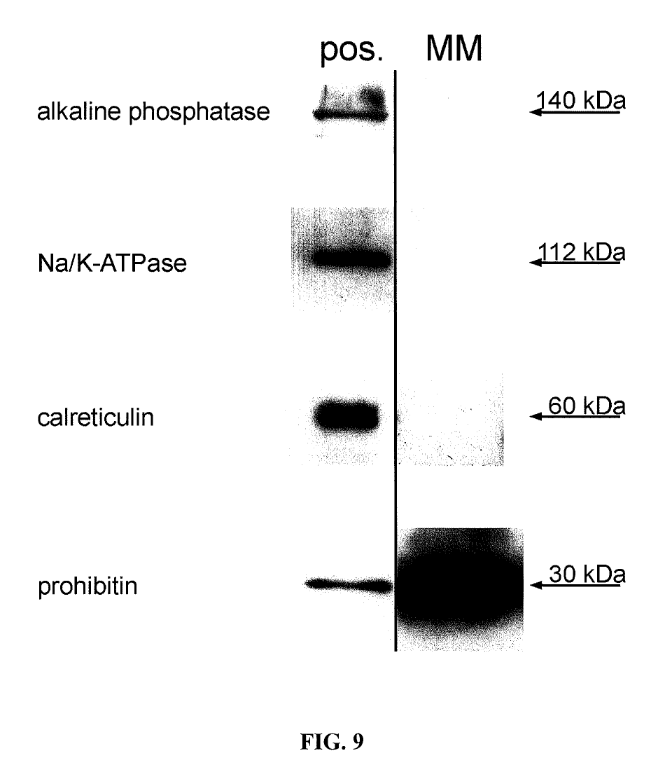

Results: The purity of the mitochondrial membrane preparations was confirmed by western blot. Negligible alkaline phosphatase, Na/K ATPase, and calreticulin were found in mitochondrial membrane preparations, which were highly enriched with prohibiton as compared to liver whole-cell lysate (FIG. 9). Densitometry was performed on the blots and confirmed these findings.

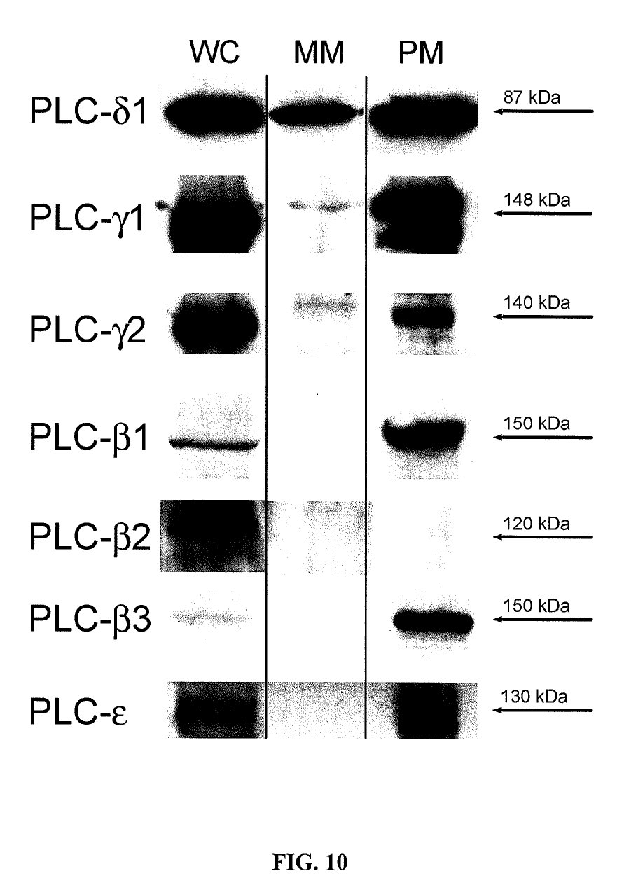

Western blot also revealed a single band at the appropriate molecular weight of approximately 88 kDa for PLC-δ1 in the isolated mitochondrial membranes, while none of the other PLC isoforms were found at substantive levels, suggesting that only PLC-61 exists in mitochondria (FIG. 10).

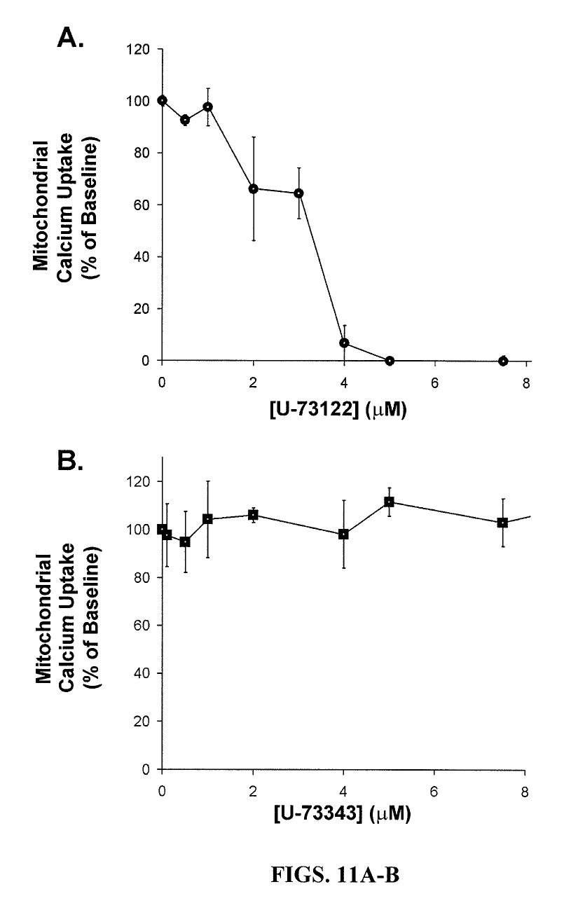

After confirming the presence of PLC-δ1 in the mitochondria, its influence on the regulation of mitochondrial calcium handling was determined using the selective PLC inhibitor U-73122. Concentrations of 5 μM or greater U-73122 inhibited mitochondrial calcium uptake by 90-100% with an EC₅₀ of 2.0-3.0 μM U-73122 (FIG. 11A). This value is consistent with the 1.0-2.1 μM EC₅₀ reported for PLC inhibition in intact cells. When tested over the same concentration range, the inactive analog U-73343 had no effect on mitochondrial calcium uptake (FIG. 11B).

Conclusions: These data identify the novel presence of phospholipase C in isolated mitochondria. Additionally, phospholipase C is shown to be a key regulator of mitochondrial calcium influx, and inhibition of phospholipase C with the selective inhibitor U-73122 completely blocks mitochondrial calcium influx.

### Example 6

Rationale: The data in Example 5 identify phospholipase C (PLC) as a previously unknown regulator of mitochondrial calcium uptake. The experiments in Example 6 were designed to determine the role of PLC in mitochondrial calcium influx in the setting of ischemia/reperfusion.

Methods: Rat livers were perfused and stored using a previously reported technique (Belous et al., 2003). Briefly, an abdominal incision was made and the portal vein was cannulated with a 20-gauge cannula. The liver was then perfused with 30 ml 4° C. University of Wisconsin solution (UW). Half of the liver was processed for calcium uptake experiments immediately, while the other half was first stored for 24 hours in 4° C. UW solution (ischemic cold storage). Mitochondria were isolated from the liver as described in Example 1. Mitochondrial calcium uptake experiments were performed on isolated mitochondria from both the ischemic and non-ischemic portions of the liver as described in Example 1. In order to simulate the cellular conditions present during ischemia/reperfusion, 1 mM ATP was added to the incubation buffer. The selective, membrane-permeable PLC inhibitor, U-73122, was added to mitochondrial calcium uptake reaction tubes to a final concentration of 5 μM. This compound was tested on mitochondria isolated from both ischemic and non-ischemic livers to determine the effect of PLC inhibition on mitochondrial calcium uptake following ischemia/reperfusion. Parallel experiments used the same concentration of the inactive analog, U-73343. Ruthenium red (10 μM) was used to block the uniporter and ensure that all observed calcium uptake was ruthenium red-sensitive, and thus entering solely through the uniporter. To simulate reperfusion, mitochondria were incubated for 30 minutes in a 37° C. water bath with ATP present, after which the total mitochondrial calcium uptake was quantified as described in Example 1.

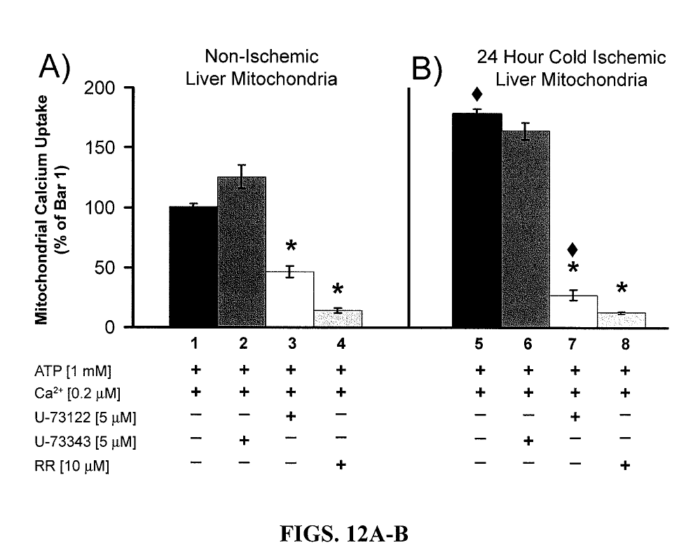

Results: Twenty-four ischemia followed by simulated reperfusion caused a 78±4% increase in mitochondrial calcium uptake (p<0.001, FIGS. 12A-B). When added to mitochondria from non-ischemic livers, U-73122 decreased mitochondrial calcium influx by 54±5% compared to mitochondria incubated with ATP and calcium alone (p<0.001). U-73343 caused a slight but significant increase in mitochondrial calcium (p=0.02) (FIG. 12A). When added to mitochondria from ischemic livers, U-73122 decreased mitochondrial calcium uptake by 85±2% compared to mitochondria from ischemic liver incubated with only ATP and calcium (p<0.001), while U-73343 caused a slight and insignificant decrease (FIG. 12B). The selective uniporter blocker, ruthenium red, prevented calcium uptake in mitochondria isolated from both ischemic and non-ischemic livers, confirming that mitochondrial calcium uptake was occurring through the uniporter (FIGS. 12A-B).

Conclusions: These data show that Phospholipase C is essential for mitochondrial calcium uptake in the setting of ischemia/reperfusion. Inhibition of mitochondrial PLC with U-73122 causes a more profound decrease in mitochondrial calcium uptake in the setting of ischemia/reperfusion than under normal conditions, suggesting that the PLC pathway may be sensitized by ischemia/reperfusion. Inhibition of mitochondrial PLC may therefore represent a novel approach to blocking mitochondrial calcium influx to protect against ischemia/reperfusion injury.

### Example 7

In further experiments, full scale liver transplants were performed in rats. Livers were harvested from male Sprague-Dawly rats and cold stored for 24 hours in UW solution, or UW containing 2-APB. The livers were then removed from cold storage and transplanted into recipient rats. The operations were performed under general anesthesia, and the animals were followed after surgery for 2 days. Mortality at 2 days was 25% in the animals that received donor livers cold stored in UW alone, and 0% for animals who received livers cold-stored in UW+2-APB, demonstrating that 2-APB offers a survival benefit in liver transplantation in rats.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. References

The following references, to the extent that they provide exemplary procedural or other details supplementary to those set forth herein, are specifically incorporated herein by reference.
U.S. Pat. No. 3,777,507
U.S. Pat. No. 3,881,990
U.S. Pat. No. 3,995,444
U.S. Pat. No. 4,186,565
U.S. Pat. No. 4,292,817
U.S. Pat. No. 4,473,637
U.S. Pat. No. 4,502,295
U.S. Pat. No. 4,504,476
U.S. Pat. No. 4,559,258
U.S. Pat. No. 4,723,974
U.S. Pat. No. 4,745,759
U.S. Pat. No. 4,766,213
U.S. Pat. No. 4,798,824
U.S. Pat. No. 4,801,599
U.S. Pat. No. 4,806,533
U.S. Pat. No. 4,935,548
U.S. Pat. No. 4,938,961
U.S. Pat. No. 4,946,851
U.S. Pat. No. 4,951,482
U.S. Pat. No. 4,963,571
U.S. Pat. No. 5,011,848
U.S. Pat. No. 5,051,432
U.S. Pat. No. 5,066,578
U.S. Pat. No. 5,157,930
U.S. Pat. No. 5,217,860
U.S. Pat. No. 5,285,657
U.S. Pat. No. 5,326,706
U.S. Pat. No. 5,370,989
U.S. Pat. No. 5,395,314
U.S. Pat. No. 5,405,742
U.S. Pat. No. 5,434,045
U.S. Pat. No. 5,459,126
U.S. Pat. No. 5,466,468
U.S. Pat. No. 5,476,763
U.S. Pat. No. 5,495,005
U.S. Pat. No. 5,552,267
U.S. Pat. No. 5,599,659
U.S. Pat. No. 5,693,462
U.S. Pat. No. 5,699,793
U.S. Pat. No. 5,719,174
U.S. Pat. No. 5,736,397
U.S. Pat. No. 5,752,929
U.S. Pat. No. 5,952,168
U.S. Pat. No. 6,013,256
U.S. Pat. No. 6,046,046
U.S. Pat. No. 6,054,261
U.S. Pat. No. 6,100,082
U.S. Pat. No. 6,187,529
U.S. Pat. No. 6,365,338
U.S. Pat. No. 6,410,743
U.S. Pat. No. 6,490,880
U.S. Pat. No. 6,492,103
U.S. Pat. No. 6,524,785
U.S. Pat. No. 6,552,083
U.S. Pat. No. 6,699,886
U.S. Publn. US2004/0106537
Amberger et al., Transplantation, 71(12):1821-1827, 2001.
Asemu et al., Am. J. Physiol. Heart Circ. Physiol., 287(6):H2598-605, 2004.
Belous et al., Transplantation, 75(10):1717-1723, 2003.
Bernard et al., J. Thorac. Cardiovasc. Surg., 90:235-242, 1985.
Gysembergh et al., Am. J. Physiol., 277(6 Pt 2):H2458-69, 1999.
Hagopian, Anal. Biochem., 273:240-251, 1999.
Kirichok et al., Nature, 427(6972):360-364, 2004.
Knox et al., Am. J. Physiol. Gastrointest. Liver Physiol., 287(3):G533-40, 2004.
Kuroda et al., Transplantation, 46(3):457-460, 1988.
Ledingham et al., Circulation, 82(Part 2)IV351-358, 1990.
Ledingham et al., J. Thorac. Cardiobasc. Surg., 93:240-246, 1987.
Lorenzo et al., Cell Death Differ., 6(6):516-524, 1999.
Menasche et al., Eur. J. Cardio. Thorax. Surg., 8:207-213, 1994.
PCT Appln. WO 94/17178
Remington's Pharmaceutical Sciences, 15^{th} Ed., pages 1035-1038 and 1570-1580, Mack Publishing Company, Easton, Pa., 1980.
Scott et al., Biochem. J., 186(1):21-33, 1980.
Wang et al., Am. J. Physiol., 263(4 Pt 1):G480-486, 1992.
Wang et al., J. Physiol., 475(3):431-437, 1994.
Wang et al., Science, 259(5098):1173-1175, 1993.
Wolf and Green, J. Biol. Chem., 274(29):20049-20052, 1999.
Zamzami et al., J. Experi. Med., 181(5):1661-1672, 1995.

What is claimed is:

1. A method of reducing acute ischemia/reperfusion injury comprising:
(a) contacting a tissue or organ with a calcium influx inhibitor prior to transplantation; and
(b) transplanting said tissue or organ into a recipient.

2. The method of claim 1, wherein said calcium influx inhibitor is:
(a) 2-APB, ruthenium red, Ru360, U-73122, or analogs or derivatives of any of these three parent compounds;
(b) any compound with the ability to inhibit mitochondrial calcium influx by directly interacting with a mitochondrial calcium influx channel controlling said calcium influx;
(c) any compound with the ability to inhibit mitochondrial calcium influx by interacting with another protein or molecule that is directly involved in regulating the activity of a mitochondrial calcium influx channel (as in, for example, mitochondrial phospholipase C).

3. The method of claim 1, further comprising the step of removing said tissue or organ from a donor.

4. The method of claim 3, wherein contacting takes place prior to removing.

5. The method of claim 3, wherein contacting takes place after removing.

6. The method of claim 3, wherein contacting takes place prior to and after removing.

7. The method of claim 1, further comprising cold or warm preservation of said tissue or organ.

8. The method of claim 7, wherein contacting takes place prior to cold or warm preservation.

9. The method of claim 7, wherein contacting takes place during cold or warm preservation.

10. The method of claim 7, wherein contacting takes place prior to and during cold or warm preservation.

11. The method of claim 1, wherein said recipient is a human.

12. The method of claim 1, wherein said recipient is a non-human mammal.

13. The method of claim 1, wherein said tissue is skin, bone, bone marrow, cartilage, cornea, skeletal muscle, cardiac muscle, cardiac valve, smooth muscle, blood vessel, a limb, or a digit.

14. The method of claim 1, wherein said organ is a kidney or portion thereof, a liver or portion thereof, a heart or a portion thereof, a pancreas or a portion thereof, a bowel or a portion thereof, or a lung or a portion thereof.

15. The method of claim 1, further comprising contacting said tissue or organ with 2-APB, ruthenium red, Ru360, U-73122 or analogs or derivatives thereof following transplantation.

16. The method of claim 1, further comprising contacting said tissue or organ with any compound with the ability to directly or indirectly inhibit mitochondrial calcium influx following transplantation.

17. The method of claim 1, further comprising administering an immunosuppressive agent to said recipient following transplantation.

18. The method of claim 1, wherein the calcium influx inhibitor is administered systemically.

19. The method of claim 1, wherein the calcium influx inhibitor is administered into the vasculature of the tissue or organ.

20. The method of claim 9, wherein said tissue or organ is immersed in a cold or warm storage solution comprising the calcium influx inhibitor.

21. A method of reducing acute ischemia/reperfusion injury during interruption of circulation to a tissue or organ to facilitate a surgical procedure, comprising:
(a) contacting a tissue or organ with a calcium influx inhibitor;
(b) interrupting the circulation to facilitate a surgical procedure;
(c) performing a surgical procedure on said tissue or organ; and
(d) restoring circulation to said tissue or organ.

22. The method of claim 21, wherein contacting takes place prior to the interruption of circulation.

23. The method of claim 21, wherein contacting takes place during the interruption of circulation.

24. The method of claim 21, comprising wherein contacting takes place after the interruption of circulation.

25. The method of claim 21, wherein contacting takes place prior to and during, during and after, or prior to and after the interruption of circulation.

26. The method of claim 21, wherein contacting takes place prior to, during and after the interruption of circulation.

27. The method of claim 21, wherein said calcium influx inhibitor is:
(a) 2-APB, ruthenium red, Ru360, U-73122 or an analog or derivative thereof;
(b) any compound with the ability to inhibit mitochondrial calcium influx by directly interacting with a mitochondrial calcium influx channel controlling said calcium influx;
(c) any compound with the ability to inhibit mitochondrial calcium influx by interacting with another protein or molecule that is directly involved in regulating the activity of a mitochondrial calcium influx channel (as in, for example, mitochondrial phospholipase C).

28. The method of claim 21, wherein said recipient is a human.

29. The method of claim 21, wherein said recipient is a non-human mammal.

30. The method of claim 21, wherein the calcium influx inhibitor is administered systemically or into the vasculature of the tissue or organ.

31. A method of reducing acute ischemia/reperfusion injury during or following sustained systemic shock or injury to a subject comprising contacting a tissue or organ in said subject with a calcium influx inhibitor.

32. The method of claim 31, wherein contacting takes place prior to the systemic shock.

33. The method of claim 31, wherein contacting takes place during the systemic shock.

34. The method of claim 31, wherein contacting takes place after the systemic shock.

35. The method of claim 31 wherein contacting takes place prior to and during, during and after, or prior to and after the systemic shock.

36. The method of claim 31, wherein contacting takes place prior to, during and after the systemic shock.

37. The method of claim 31, wherein said calcium influx inhibitor is 2-APB, ruthenium red, Ru360, U-73122 or an analog or derivative thereof.

38. The method of claim 31, wherein said recipient is a human.

39. The method of claim 31, wherein said recipient is a non-human mammal.

40. The method of claim 31, wherein the calcium influx inhibitor is administered systemically or into the vasculature of the tissue or organ.

41. The method of claim 31, wherein the systemic shock is selected from:
(a) cardiogenic shock, including chronic heart failure, acute myocardial infarction, or following cardiac surgery;
(b) hypovolemic shock, including hemorrhage, acute trauma, or dehydration;
(c) obstructive shock, including pulmonary embolism or pericardial tamponade; and
(d) distributive shock, as in, for example, sepsis, anaphylaxis, or spinal shock.
